(19) United States
(12) Patent Application Publication
(10) Pub. No.: US 2023/0145064 A1
(43) Pub. Date: May 11, 2023

(54) VARIABLE-BANDWIDTH TRANSDUCERS WITH ASYMMETRIC FEATURES

(71) Applicants: Kobi VORTMAN, Haifa (IL); Yoav LEVY, Hinanit (IL); Oleg PRUS, Haifa (IL)

(72) Inventors: Kobi VORTMAN, Haifa (IL); Yoav LEVY, Hinanit (IL); Oleg PRUS, Haifa (IL)

(21) Appl. No.: 17/520,068

(22) Filed: Nov. 5, 2021

(51) Int. Cl. A61B 8/00; A61B 8/08; B06B 1/02

(52) U.S. Cl. CPC A61B 8/4494; A61B 8/0858; A61B 8/546; B06B 1/0207; B06B 2201/76

(57) ABSTRACT

Transducer elements have one or more asymmetric features that give rise to multiple natural resonance frequencies. The feature(s) can be discrete (e.g., steps, bars, or gemstone-like facets) or continuous across one or more dimensions of the transducer element (e.g., a triangular prism). A transducer element can be driven at more than one resonance frequency; multiple frequencies will excite more than one feature in parallel, each producing an output emission with a characteristic frequency and phase. An optimal frequency—i.e., one that maximizes the peak acoustic intensity or acoustic power at the target—within a certain frequency range may be determined, and a plurality of asymmetric transducer elements may be driven at a center frequency that coincides with or is close to this optimal frequency.

### TECHNICAL FIELD

The present invention relates, generally, to focused-ultrasound equipment, and more particularly to transducer configurations for with variable output profiles.

### BACKGROUND

Focused ultrasound (i.e., acoustic waves having a frequency greater than about 20 kHz) can be used to image or therapeutically treat internal body tissues within a patient. For example, ultrasonic waves may be used to ablate tumors, eliminating the need for the patient to undergo surgery. For this purpose, a piezo-ceramic transducer is placed external to the patient, but in close proximity to the internal tissue to be ablated or otherwise treated (the “target”). The transducer converts an electronic drive signal into mechanical vibrations, resulting in the emission of acoustic waves (a process hereinafter referred to as “sonication”). The transducer may be shaped so that the waves converge in a focal zone. Alternatively or additionally, the transducer may be formed by a plurality of individually driven transducer elements whose phases (and, optionally, amplitudes) can each be controlled independently from one another and, thus, can be set so as to result in constructive interference of the individual acoustic waves in the focal zone. Such a “phased-array” transducer facilitates steering the focal zone to different volumetric locations by adjusting the relative phases between the transducer elements. Magnetic resonance imaging (MRI) may be utilized to visualize the focus and target in order to guide the ultrasound beam.

FIG. 1 illustrates an exemplary focused-ultrasound system 100. The system 100 includes a transducer array 101 having multiple ultrasound transducer elements 102, which are arranged in an array at the surface of a housing 104. The array 101 may comprise a single row or a matrix, or generally any 3D arrangement, of transducer elements 102. The array 101 may have a curved (e.g., spherical or parabolic) shape, as illustrated, or may include one or more planar or otherwise shaped sections. Its dimensions may vary, depending on the application, between millimeters and tens of centimeters. The transducer elements 102 may be piezoelectric ceramic elements, or be made of piezo-composite materials, or any other material structure capable of converting electrical energy to acoustic energy. To damp the mechanical coupling between the elements 102, they may be mounted on the housing 104 using silicone rubber or any other suitable damping material or may be laterally separated mechanically (e.g., air spacing).

The transducer elements 102 are driven via separate drive channels by a controller 106. For n transducer elements 102, the controller 106 may contain n control circuits each comprising an amplifier and a phase control circuit, with each control circuit driving one of the transducer elements 102. The controller 106 may split a radio-frequency (RF) input signal, typically in the range of 0.1 MHz to 10 MHz, into n channels for the n control circuits. In conventional systems, the controller 106 is configured to drive the individual transducer elements 102 of the array at the same frequency, but at different phases and different amplitudes so that they collectively produce a focused ultrasound beam at a desired location. The controller 106 desirably provides computational functionality, which may be implemented in software, hardware, firmware, hardwiring, or any combination thereof, to compute the required phases and amplitudes for a desired focus location; these phase/amplitude computations may include corrections that compensate for aberrations resulting from ultrasound reflection or refraction at tissue interfaces or propagation in tissue having various acoustic parameters, which may be determined based, e.g., on computer tomography (CT) and/or MM or other images of the anatomical region of interest. In general, the controller 106 may include several separable apparatus, such as a frequency generator, a beamformer containing the amplifier and phase control circuitry, and a computer (e.g., a general-purpose computer) performing the computations and communicating the phases and amplitudes for the individual transducer elements 102 to the beamformer. Such systems are readily available or can be implemented without undue experimentation.

The system 100 may further include an MRI apparatus 108 in communication with the controller 106 for performing MRI-guided focused ultrasound treatment. An exemplary apparatus 108 is illustrated in more detail in FIG. 2. The apparatus 108 may include a cylindrical electromagnet 204, which generates a static magnetic field within a bore 206 of the electromagnet 204. During medical procedures, a patient is positioned inside the bore 206 on a movable support table 208. A region of interest 210 within the patient (for example, the patient's head) may be positioned within an optimal imaging region 212 wherein the magnetic field is substantially homogeneous. An RF transmitter coil 214 surrounding the imaging region 212 emits RF pulses into the imaging region 212, and receives MR response signals emitted from the region of interest 210. The MR response signals are amplified, conditioned, and digitized into raw data using an image-processing system 216, and further transformed into arrays of image data by methods known to those of ordinary skill in the art. Based on the image data, a treatment region (e.g., a tumor) is identified. The ultrasound phased array 220, disposed within the bore 206 of the MM apparatus and, in some embodiments, within the imaging region 212, is then driven so as to focus ultrasound into the treatment region. The MRI apparatus 108 facilitates visualizing the focus 112 based on the effect of sonication on the target tissue. For example, any of a variety of MRI-based thermometry methods may be employed to observe the temperature increase resulting from ultrasound absorption in the focus region. Alternatively, MR-based acoustic radiation force imaging (ARFI) may be used to measure the tissue displacement in the focus. Such measurements of the focus can be used as feedback for driving the ultrasound transducer array 220 so as to maximize the peak intensity.

The goal of focused-ultrasound treatment is generally to effectively deposit maximal acoustic energy at the target while minimizing the exposure of healthy tissue surrounding the target, as well as tissues along the path between the transducer and the target, to ultrasound. The deposited acoustic energy generally correlates to the peak intensity or acoustic power of the focused beam and may cause heating and/or vibration of the target tissue. The treatment effects (e.g., the temperature increase) of the ultrasound procedure may be optimized by maximizing the peak acoustic intensity or power of the focused ultrasound beam at the target.

In the conventional ultrasound system 100, the transducer elements 102 in the array 101 are generally “tiled” to form a flat or curved surface. Each transducer element 102 is typically a square or rectangular piezoelectric block whose dimensions and the transducer thickness dictate the natural resonance frequency of the element—i.e., its single center frequency. Although the elements 102 can be driven to generate acoustic energy over a range of frequencies, greatest efficiency and peak power is attained when the element is driven at its natural resonance frequency. The output bandwidth profile of a conventional transducer element is narrow, so efficiency drops quickly as the driving frequency diverges from the natural resonance frequency. This may limit the practical ability to achieve high energy deposition at the focus outside a narrow range of operating frequencies. Moreover, because the ability to steer the acoustic beam may depend on the size and the number of the transducer elements 102 in the transducer array 101 and the peak intensity of the focused beam may depend on the steering angle, the fixed, non-adjustable configuration of the elements 102 may limit steering, and therefore, once again, the peak intensity of the focused beam.

Accordingly, there is a need for transducer elements that exhibit more than one center frequency and systems capable of exploiting such transducer elements to optimize energy delivery and steering in ultrasound applications.

### SUMMARY

Embodiments of the present invention utilize transducer elements with one or more asymmetric features that give rise to multiple natural resonance frequencies. The feature(s) can be discrete (e.g., steps, bars, or gemstone-like facets) or continuous across one or more dimensions of the transducer element (e.g., a triangular prism). A transducer element can be driven at more than one resonance frequency; multiple frequencies will excite more than one feature in parallel, each producing an output emission with a characteristic frequency and phase. In general, fewer features result in sharper resonance peaks with fewer (or smaller) side lobes.

In one application, as described in U.S. Patent Publ. No. 2020/0205782, the entire disclosure of which is hereby incorporated by reference, an optimal frequency—i.e., one that maximizes the peak acoustic intensity or acoustic power at the target—within a certain frequency range is determined. The asymmetric transducer elements are driven at a center frequency that coincides with or is close to this optimal frequency. The terms “optimal,” “optimizing,” “maximum,” “maximizing”, etc. may generally involve a substantial improvement (e.g., by more than 10%, more than 20%, or more than 30%) over the prior art, but do not necessarily imply achievement of the best theoretically possible frequency, energy absorption, etc. Rather, optimizing the frequency, or maximizing the acoustic intensity/power at the target, involves selecting the best frequency practically discernible within the limitations of the utilized technology and method.

Accordingly, in a first aspect, the invention relates to an ultrasound emitter comprising, in various embodiments, a piezoelectric block having at least one asymmetric feature producing a plurality of natural resonance frequencies; and a driver for driving the piezoelectric block at one or more of the natural resonance frequencies to produce an ultrasound emission having a dominant output frequency and phase dictated by the at least one asymmetric feature. In some embodiments, the asymmetric feature(s) is/are continuous along at least one axis of the piezoelectric block; for example, the piezoelectric block may be a triangular prism. The asymmetric feature(s) may be discrete, e.g., a series of steps along an axis of the piezoelectric block or other configuration of raised features having different heights relative to a flat surface of the piezoelectric block. In some embodiments, the asymmetric feature(s) is/are associated with specific geometric location.

In a second aspect, the invention pertains to a system for delivering ultrasound energy to a target region. In various embodiments, the system comprises an ultrasound transducer comprising a plurality of transducer elements for generating a focal zone of acoustic energy at the target region, wherein at least some of the transducer elements comprise a piezoelectric block having at least one asymmetric feature producing a plurality of natural resonance frequencies; at least one driver circuit connected to the transducer elements; and a controller configured to determine an optimal sonication frequency for maximizing a peak acoustic intensity in the focal zone; and based at least in part on the determined optimal sonication frequency, activate the driver circuit(s) to drive the transducer elements with asymmetric features at one or more of the natural resonance frequencies to produce an ultrasound emission having a dominant output frequency corresponding to the optimal sonication frequency.

In some embodiments, the system further includes an imaging system for acquiring images of the target region or a non-target region located between the transducer and the target region. For example, the imaging system may comprises one or more of of a computer tomography (CT) device, a magnetic resonance imaging device (MRI), a positron emission tomography (PET) device, a single-photon emission computed tomography (SPECT) device, or an ultrasonography device.

The controller may be further configured to determine, based at least in part on the acquired images, a spatial configuration of the target region with respect to the transducer. The spatial configuration may comprise an orientation and/or a location.

In various embodiments, the controller is further configured to compute a steering angle of the focal zone based at least in part on the spatial configuration of the target region with respect to the transducer. Alternatively or in addition, the controller may be further configured to determine a plurality of suboptimal frequencies, each associated with a parameter, wherein (i) a change in the parameter results in a change in the peak acoustic intensity in the focal zone and (ii) the suboptimal frequency corresponds to a maximum of the peak acoustic intensity resulting from changes in the associated parameter; and to determine the optimal sonication frequency based at least in part on the suboptimal frequencies. The controller may be configured to assign a weighting factor to each of the suboptimal frequencies and determine the optimal sonication frequency based at least in part on the weighting factors. The controller may assign the weighting factors based on a first anatomic characteristic of the target region, a second anatomic characteristic of a non-target region located between the transducer and the target region, a steering angle of the focal zone, a contribution of each parameter to the maximum of the peak acoustic intensity, and/or retrospective data based on study of patients who have undergone ultrasound treatment. The first or the second anatomic characteristic may include or consist of a tissue type, a tissue property, a tissue structure, a tissue thickness and/or a tissue density. Alternatively or in addition, the controller may be configured to assign the weighting factors using a machine-learning or evolutionary approach.

In some embodiments, the controller is further configured to determine a second one of the suboptimal frequencies based at least in part on a first one of the suboptimal frequencies. The controller may be further configured to use a physical model to predict a thermal map of the target region and non-target region based at least in part on the acquired images; and determine the optimal sonication frequency based at least in part on the predicted thermal map.

As used herein, the terms “approximately,” “roughly,” and “substantially” mean±10%, and in some embodiments, ±5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

The foregoing and the following detailed description will be more readily understood when taken in conjunction with the drawings, in which:

FIG. 1 schematically illustrates an exemplary focused ultrasound system in accordance with various embodiments.

FIG. 2 illustrates an Mill system in accordance with various embodiments.

FIGS. 3A-3C illustrate, respectively, perspective and elevational views of a stepped transducer configuration in accordance with embodiments of the present invention, and a polar plot of output amplitude as a function of driving frequency.

FIGS. 4A-4C illustrate, respectively, perspective and elevational views of a transducer shaped as a triangular prism (i.e., a wedge) in accordance with embodiments of the present invention, and a polar plot of output amplitude as a function of driving frequency.

FIGS. 5A and 5B illustrate different strategies for coupling driver circuitry to a transducer element.

FIG. 6 is a block diagram illustrating a system for maximizing the peak intensity/power of a focused beam at the target region in accordance with various embodiments.

### DETAILED DESCRIPTION

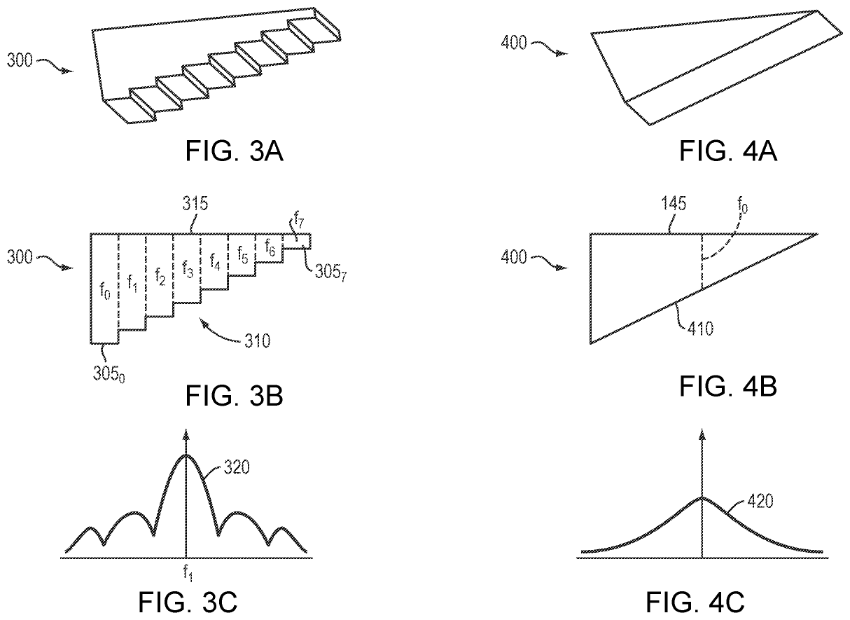

Various embodiments hereof provide transducer elements that, by virtue of one or more asymmetric features, exhibit more than one natural resonance frequency. FIGS. 3A-3C illustrate a transducer element 300 having a plurality of discrete features, namely, steps 305₀ . . . 305₇ along the lower surface 310 of the element 300. One or more electrodes may be plated or otherwise applied to the flat top surface 315 as described in greater detail below. Each of the steps 305 may correspond to a different natural resonance frequency f₀ . . . f₇. It should be understood, however, that although the segments through the element 300 defined by the steps 305 are associated with different resonance frequencies, the segments are not isolated from each other and are instead regions of the continuous bulk material; hence, the different resonance frequencies are not dictated solely by the rectangular region of material within each illustrated segment. Nonetheless, because of the discrete character of the features 305, the natural resonance frequencies are also discrete. Of course, it is possible, if desired, to create the transducer element 300 from separate blocks that are joined together with acoustic isolation between them. For reasons that will become clear, this is generally not a preferred approach.

The transducer element 300 is typically made of a piezoelectric ceramic, or may be a piezo-composite material or any other material capable of converting electrical energy to acoustic energy. A common piezoelectric ceramic material is PZT, or lead zirconate titanate (Pb[ZrₓTi₁₋ₓ)]O₃), which may be combined with a polymer as a composite. When the element 315 is driven at one of the natural resonance frequencies—e.g., f₃—the acoustic output exhibits a peak 320 as illustrated in FIG. 3C with side lobes of diminishing amplitude. The side lobe frequencies are determined by the geometry of the steps 305. The fewer the number of steps 305, the fewer and smaller (in amplitude) the side lobes will be, and consequently, the sharper and taller the peak 320 will be. Accordingly, the number of asymmetric features in a transducer element represents a trade-off between the number of addressable center frequencies and the peak output the element is capable of delivering.

At the extreme, as illustrated in FIGS. 4A-4C, the asymmetric feature of the transducer element 400 is a continuous angled surface 410 that eliminates the rectangular symmetry of the element 400 along one axis; in effect, the surface 410 represents an infinite sequence of steps and so does not create a discrete sequence of natural resonance frequencies, although at any particular driving frequency f₀, one natural resonance frequency will dominate and produce a shallow peak amplitude 420.

More generally, the transducer element may have any shape with arbitrary features to produce a desired number of center frequencies with maximum peak and minimum side lobe amplitudes. These features may be recesses, facets, curved regions, etc. according to the desired behavior. A typical transducer includes, in addition to the active piezo block, an impedance-matching layer on one side and a backing material on the other side. The backing material determines the overall damping characteristics of the transducer, and typically has an impedance similar to that of the active block to maximize the output bandwidth.

Factors apart from feature type and size that influence resonant frequency include the thickness of the piezo block (i.e., the average distance between the surfaces 310, 410 and 315, 415); the thickness and density of matching layer; the lateral (i.e., non-thickness) dimensions of the piezo block; the specific piezo material used; the type and thickness of any protective coating utilized; and the degree of impedance matching with the driving electronics.

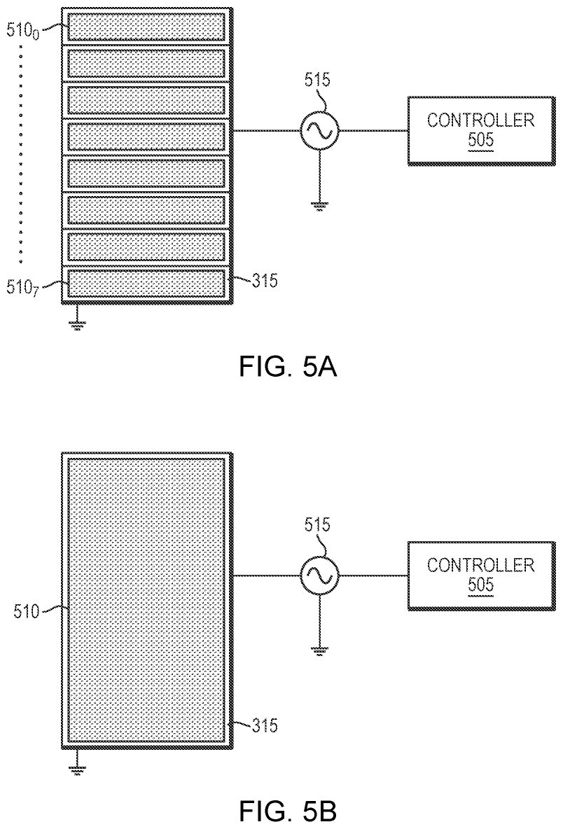

For transducer elements with discrete features, it is possible to drive separate portions of the transducer element body separately. As illustrated in FIG. 5A, a controller 505 may drive one (or more) of series of separate electrode contacts 510₀ . . . 510₇ using an AC source 515. The contacts 510 may be aluminum, titanium or other suitable conductive material, and may be plated on or affixed to the surface 315 of the transducer element 300 (see FIG. 3B) so as to overlie the stepped features corresponding to frequencies f₀ . . . f₇. Although this configuration is useful for transducer elements assembled from acoustically isolated segments, it is generally unnecessary when the element 300 is a unitary block of material. In such cases, a single analog control line may connect the controller 505 to a unitary electrode contact 510, and different frequencies generated by the AC source 515 will preferentially excite the element's closest natural resonance frequency. Hence, it is possible to exploit different resonance frequencies of a unitary block element without internal isolation.

As described in the '5782 application mentioned above, treatment planning can identify an optimal frequency for sonication that maximizes energy deposition at a target given the beam steering necessary for the various ultrasound beams to converge thereat. For example, treatment planning may begin with determining suboptimal frequencies that are used as the basis for identifying an optimal frequency. The first suboptimal frequency, fⱼ (or the first range of suboptimal frequencies), may be obtained with respect to a single parameter (e.g., energy absorption of the acoustic energy at the target region). Thus, sonications applied at the suboptimal frequency fⱼ generate maximal energy absorption at the target while minimizing energy deposition in the non-target region. Within the focal zone, the acoustic power of the beam is (at least partially) absorbed by the tissue, thereby generating heat and raising the temperature of the tissue to a point where the cells are denatured and/or ablated. Excessive energy absorption by the non-target tissue in the beam path zone may, however, cause damage thereto. As a result, the choice of the ultrasound frequency reflects a trade-off between absorption of the acoustic energy along the beam path and absorption at the target; the suboptimal frequency, fⱼ (or the suboptimal frequency range), is preferably selected to provide maximal energy absorption at the target while avoiding overheating tissue in the beam path zone.

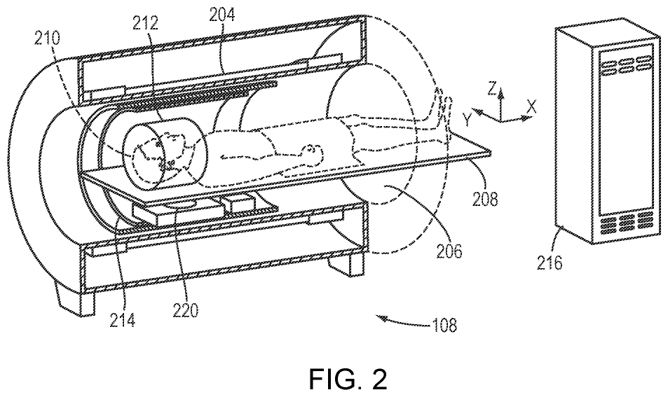

Identifying suboptimal frequencies and, ultimately, the optimal frequency for sonication may be accomplished empirically or using a physical model. In the former case, one or more imaging apparatus is activated to acquire images of the patient's anatomy within a region of interest. The images may be 3D images or a set of 2D image slices suitable for reconstructing 3D images of the anatomic region of interest. The imaging device may include, for example, the MRI apparatus 108 (as depicted in FIG. 2), a computer tomography (CT) device, a positron emission tomography (PET) device, a single-photon emission computed tomography (SPECT) device, or an ultrasonography device. The images are processed by a controller to identify therein the location of the target and/or non-target regions, enabling computation of the relative phases and/or amplitude settings of some or all of the ultrasound transducer elements to produce a beam focused at the identified target region. This step may utilize a physical model, taking into account the geometry as well as the position and orientation of the ultrasound transducer relative to the target region, as well as any a-priori knowledge and/or image-derived information about the intervening tissues. Based on the relative phase and/or amplitude settings of the ultrasound transducer elements and the anatomic and/or material characteristics of the target/non-target tissue, the physical model may computationally predict ultrasound energy delivered to the target region and/or non-target regions at a specific ultrasound frequency, the conversion of ultrasound energy or pressure into heat and/or tissue displacement at the target region and/or non-target regions, and/or the propagation of the induced effects through the tissue.

A second suboptimal frequency, f that minimizes the energy attenuation of the focused beam propagating toward the target region resulting from a specific steering angle may then be determined. The “steering angle” of any one transducer element of the array is the angle between the first focal axis extending generally orthogonally from the element to an “unsteered” focal zone at which the element contributes the maximum possible power, and a second focal axis extending from the transducer element to a “steered-to” focal zone located at the target region. The “steering ability” of the transducer array is defined as the steering angle at which energy delivered to the steered-to focal zone falls to half of the maximum power delivered to the unsteered focal zone. Notably, the steering angle of each transducer element of a phased array may be different, but as the distance from the elements to the focal zone increases, the respective steering angles for the array elements approach the same value. In practice, because the distance between the transducer array and the target region is sufficiently longer than the distance between the transducer elements, the steering angles associated with the transducer elements in the array can be considered the same.

To determine the suboptimal frequency fᵢ that minimizes the energy attenuation of the steering beam at the focal zone having a specific steering angle, the steering angle of the focal zone at the target is first computed based on the spatial arrangement (e.g., positions and orientations) of the ultrasound transducer elements with respect to the target region. Ultrasound waves/pulses each having a test frequency in a test range are computationally applied to sonicate the target region. The physical model may then predict energy attenuation of the focused beam propagating through the intervening tissue to the target region associated with the computed steering angle. Because the acoustic power at the focal zone depends on the steering angle and the sonication frequency, the physical model may vary the sonication frequency and predict energy attenuation associated with the updated frequency at the steering angle. The test frequency that corresponds to the minimal energy attenuation at the determined steering angle is identified as the suboptimal frequency fᵢ.

Accordingly, the treatment planner has taken into account at least two parameters that may significantly affect the acoustic power or peak intensity at the target region—i.e., the anatomic/material characteristics of the target and/or non-target tissue (by determining the suboptimal frequency fⱼ associated with maximal energy absorption at the target region) and the specific steering angle of the focal zone (by determining the suboptimal frequency fᵢ associated with minimal energy attenuation of the focused beam traversing the intervening tissue). The two suboptimal frequencies fⱼ and fᵢ may or may not be the same. If they are different, choice of the optimal sonication frequency reflects a trade-off among the absorption of the acoustic energy in the path zone, the power absorption at the target, and the energy attenuation resulting from beam propagation at a specific steering angle.

The treatment planner may then, based on the determined suboptimal frequencies fⱼ and fᵢ, determine an optimal frequency f that maximizes the acoustic peak intensity at the target region. For example, if the second suboptimal frequency fᵢ is determined by varying the frequency over the test range between fⱼ—Δf and fⱼ+Δf, and fᵢ corresponds to the minimal energy attenuation at the specific steering angle within this test range, the planner may determine that the optimal frequency f is fᵢ. Alternatively, the treatment planner may assign a weighting factor to each of the suboptimal frequencies fⱼ and fᵢ and then determine the optimal frequency f based on the weighted average thereof. The weighting factors may be assigned based on, for example, the tissue type of the target and/or non-target tissue, the steering angle, prior knowledge, and the degree of impact on the parameter (e.g., energy absorption at the target and/or energy attenuation at the specific angle) resulting from the change of the frequency. Generally, a larger impact indicates that the suboptimal frequency associated therewith is more important for achieving the maximal peak intensity/power at the target region; thus, a larger weighting factor may be assigned thereto. For example, when adjusting the sonication frequency results in a significant decrease in energy absorption at the target region but only minor increase of energy attenuation at the steering angle of the focused beam onto the target region, the treatment planner may assign a larger weighting factor to the frequency fⱼ (that takes into account the energy absorption in the beam path zone and target region) and a smaller weighting factor to the frequency fᵢ (that takes into account the energy attenuation at the steering angle) for computing the optimal frequency f. Conversely, if adjustment of the sonication frequency results in a significant increase of the energy attenuation at the specific steering angle of the focal zone, a larger weighting factor may be assigned to the frequency fᵢ.

In some embodiments, the tissue types and their associated absorption coefficients (or attenuation coefficients) and the steering angles of the focal zone and their associated energy attenuations at relevant frequencies (e.g., frequencies suitable for ultrasound treatment) may be obtained empirically prior to and/or during ultrasound treatment, using numerical simulations (e.g., implementing the physical model) and/or based on known literature; this information may be stored as a lookup table in a database and may be retrieved when determining the weighting factors assigned to the frequencies fⱼ and fᵢ. Additionally or alternatively, the weighting factors of the frequencies fⱼ and fᵢ may be assigned based on a retrospective study of the patients who have undergone the ultrasound treatment procedures, or may be obtained using a conventional learning or evolutionary algorithm.

Various techniques can be used to measure the acoustic intensity/power in the target—directly or indirectly via a related physical quantity—to then maximize the peak intensity/power via selection of the optimal frequency f. One approach is to monitor the temperature at the target, which increases proportionally to the amount of acoustic energy deposited therein. Thermometry methods may be based, e.g., on MRI, and may utilize a system such as that depicted in FIG. 2, in conjunction with suitable image-processing software. Among various methods available for MR thermometry, the proton resonance frequency (PRF) shift method is often the method of choice due to its excellent linearity with respect to temperature change, near-independence from tissue type, and temperature map acquisition with high spatial and temporal resolution. The PRF shift method exploits the phenomenon that the MR resonance frequency of protons in water molecules changes linearly with temperature. Since the frequency change with temperature is small, only −0.01 ppm/° C. for bulk water and approximately −0.0096 to −0.013 ppm/° C. in tissue, the PRF shift is typically detected with a phase-sensitive imaging method in which the imaging is performed twice: first to acquire a baseline PRF phase image prior to a temperature change and then to acquire a second phase image after the temperature change, thereby capturing a small phase change that is proportional to the change in temperature. A map of temperature changes may then be computed from the MR images by determining, on a pixel-by-pixel basis, phase differences between the baseline image and the treatment image, and converting the phase differences into temperature differences based on the PRF temperature dependence while taking into account imaging parameters such as the strength of the static magnetic field and echo time (TE) (e.g., of a gradient-recalled echo). Various alternatives or advanced methods may be used to compensate for patient motion, magnetic-field drifts, and other factors that affect the accuracy of PRF-based temperature measurements; suitable methods known to those of skill in the art include, e.g., multibaseline and referenceless thermometry.

Using a PRF-based or any other suitable thermometry method, the optimal ultrasound frequency within a specified range can be determined by driving the transducer successively at a number of different frequencies (e.g., at specified frequency intervals within the selected range), while keeping the power and duration (or, more generally, the total transmitted energy) the same, to focus ultrasound at the target site of a particular patient, and measuring the temperature increase at the target for each such sonication. This is done prior to treatment; thus, in order to avoid tissue damage, the ultrasound transducer is driven at much lower power than subsequently during treatment (while being high enough to obtain meaningful signals). Further, to ensure the comparability of the measurements for different frequencies, each temperature increase is preferably measured against a similar baseline temperature. This can be accomplished by waiting a sufficient amount of time following each sonication to let the tissue cool back down to a temperature approximately equal to the baseline temperature and using sufficiently low energy that effects on the tissue due to temperature changes are limited (e.g., clinically insignificant). When the temperature increase has been measured at the various discrete frequencies within the range of interest, the frequency for which the temperature increase is maximum is selected for operating the transducer during subsequent treatment.

Another quantity usefully related to ultrasound energy absorption in tissue is the temporary local displacement of that tissue due to acoustic radiation pressure, which is highest at the focus (where the waves converge and highest intensity is achieved). The ultrasound pressure creates a force that displaces the tissues in a way that directly reflects the acoustic field. The displacement field can be visualized, using a technique such as MR-ARFI, by applying transient-motion or displacement-sensitizing magnetic field gradients to the imaging region by gradient coils, which are part of standard MRI apparatus (such as apparatus 108 depicted in FIG. 2) and are typically located near the cylindrical electromagnet 204. When the ultrasound pulse is applied in the presence of such gradients, the resulting displacement is directly encoded into the phase of the MR response signal. For example, the gradient coils and transducer may be configured such that the ultrasound pulse pushes material near the focus toward regions of the magnetic field with higher field strengths. In response to the resulting change in the magnetic field, the phase of the MR response signal changes proportionally, thereby encoding in the signal the displacement caused by the ultrasound radiation pressure. Further detail about MR-ARFI is provided in U.S. Pat. No. 8,932,237, the entire disclosure of which is hereby incorporated herein by reference.

Accordingly, various embodiments of the present invention provide approaches for optimizing the ultrasound frequency so as to achieve the treatment goal—i.e., maximizing the peak acoustic intensity/power at the target while minimizing the exposure of non-target tissue to ultrasound. Because the peak acoustic intensity may depend on multiple parameters (such as absorption of the acoustic beam at the target tissue and non-target tissue in the beam path zone, the steering angle, and the focal area of the focal zone, etc.) that are frequency dependent, some embodiments sequentially evaluate each of these parameters and determine the suboptimal frequency associated therewith; the optimal frequency for treatment is then determined from these suboptimal frequencies. For example, each suboptimal frequency may be assigned a weighting factor corresponding to its contribution toward the desired treatment goal; the optimal frequency can then be computed as the weighted sum of the suboptimal frequencies. Alternatively, the treatment planner may evaluate these parameters simultaneously and then numerically determine the optimal frequency by assigning each parameter-associated suboptimal frequency with a weighting factor based on its importance for achieving the treatment goal as described above.

It should be noted that the approaches for determining the optimal ultrasound frequency f described herein are presented as representative examples only; any other approaches involving evaluating multiple parameters affecting the peak acoustic intensity/power at the target region and then determining the optimal sonication frequency based on the evaluation may be implemented and are thus within the scope of the present invention. In addition, the frequency optimization may be based other parameters, such as the simulated thermal map of the target/non-target regions during treatment, the resonance frequency of microbubbles, etc.

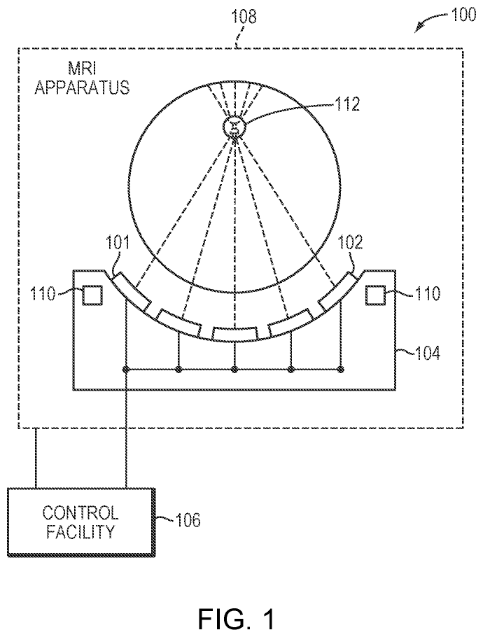

The treatment planner utilized in the treatment-planning approach described above can be implemented in any suitable combination of hardware, software, firmware, or hardwiring in conjunction with one or more ultrasound transducers and imaging apparatus (e.g., an MM apparatus) for measuring the peak intensity/power at the focus, or another parameter indicative thereof. The combination of hardware, software, firmware, or hardwiring may be integrated with the ultrasound controller (e.g., controller 106 of FIG. 1) and/or the imaging apparatus or other device for measuring peak acoustic intensity/power at the target (e.g., the image-processing system 216 shown in FIG. 2), or provided as a separate device in communication therewith.

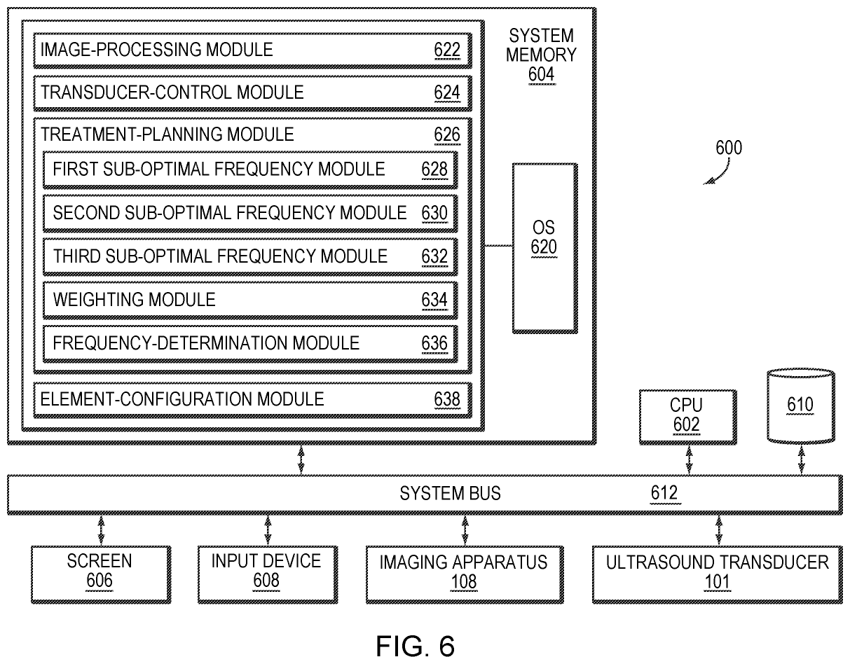

In some embodiments, the controller is implemented with a suitably programmed general-purpose computer; FIG. 6 shows an exemplary embodiment. The computer 600 includes one or more processors 602 (e.g., a CPU) and associated system memory 604 (e.g., RAM, ROM, and/or flash memory), user input/output devices (such as a screen 606 and a keyboard, mouse, etc. 608), and typically one or more (typically non-volatile) storage media 610 (e.g., a hard disk, CCD, DVD, USB memory key, etc.) and associates drives. The various components may communicate with each other and with external devices (such as the ultrasound transducer and/or the imaging apparatus) via one or more system buses 612.

The system memory 604 contains instructions, conceptually illustrated as a group of modules, that control the operation of the processor 602 and its interaction with the other hardware components. An operating system 620 directs the execution of low-level, basic system functions such as memory allocation, file management and operation of the peripheral devices. At a higher level, one or more service applications provide the computational functionality required for the treatment planner to determine the optimal frequency in accordance herewith. For example, as illustrated, the system may include an image-processing module 622 that allows analyzing images from the MRI (or other imaging) apparatus to identify the target therein and visualize the focus to ensure that it coincides with the target; a transducer-control module 624 for computing the relative phases and amplitudes of the transducer elements based on the target location as well as for controlling ultrasound-transducer operation during both frequency optimization and treatment; and a treatment-planning module 626 providing the computational functionality required for frequency optimization (e.g., acquiring data about the frequency-dependence of the peak acoustic intensity or power at the target and selecting an optimum frequency (or multiple respective optimum frequencies for various transducer segments) based thereon) as described above. More specifically, a first submodule 628 may determine the first suboptimal frequency fⱼ corresponding to maximal energy absorption at the target; a second submodule 630 may determine the second suboptimal frequency fⱼ corresponding to minimal energy attenuation of the focused beam propagating through the intervening tissue before reaching the target; a third submodule 632 may determine one or more suboptimal frequencies corresponding to other parameters affecting the peak acoustic intensity/power; a weighting module 634 may assign the weighting factors to the various suboptimal frequencies in the manner described in detail above; and a frequency-determine module 636 for determining the optimal frequency based on the suboptimal frequencies. The treatment-planning module 626 may be in communication with the image-processing module 622 to acquire information of the target/non-target regions obtained from the images and/or the transducer-control module 624 for providing the determined optimal frequency thereto so as to operate the transducer in accordance therewith. In addition, the system may include an element-configuration module 638 for determining the configurations (e.g., sizes and/or shapes) of the transducer elements at a specific steering angle for improved acoustic intensity/power; the transducer-control module 624 may then be responsive to the element-configuration module 638 and/or the treatment-planning module 626 for cause the transducer to sonicate the target in accordance with the determined configurations and optimal frequency.

Of course, the depicted organization of the computational functionality into various modules is but one possible way to group software functions; as a person of skill in the art will readily appreciate, fewer, more, or different modules may be used to facilitate frequency-optimization in accordance herewith. However grouped and organized, software may be programmed in any of a variety of suitable programming languages, including, without limitation, PYTHON, FORTRAN, PASCAL, JAVA, C, C++, C#, BASIC or combinations thereof. Furthermore, as an alternative to software instructions executed by a general-purpose processor, some or all of the functionality may be provided with programmable or hard-wired custom circuitry, including, e.g., a digital signal processor, programmable gate array, application-specific integrated circuit, etc.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. For example, instead of MR-based thermometry or ARFI, any non-invasive imaging technique capable of measuring the (physical or therapeutic) effect of the acoustic beam at the focus may generally be used to select an optimal frequency (or multiple optimal frequencies for different segments) in accordance herewith. Accordingly, the described embodiments are to be considered in all respects as only illustrative and not restrictive.

What is claimed is:

1. An ultrasound emitter comprising:
a piezoelectric block having at least one asymmetric feature producing a plurality of natural resonance frequencies; and
a driver for driving the piezoelectric block at one or more of the natural resonance frequencies to produce an ultrasound emission having a dominant output frequency and phase dictated by the at least one asymmetric feature.

2. The emitter of claim 1, wherein the at least one asymmetric feature is continuous along at least one axis of the piezoelectric block.

3. The emitter of claim 2, wherein the piezoelectric block is a triangular prism.

4. The emitter of claim 1, wherein the at least one asymmetric feature is discrete.

5. The emitter of claim 1, wherein the at least one asymmetric feature is a series of steps along an axis of the piezoelectric block.

6. The emitter of claim 1, wherein the at least one asymmetric feature is a plurality of raised features having different heights relative to a flat surface of the piezoelectric block.

7. The emitter of claim 1, wherein the at least one asymmetric feature is associated with specific geometric location.

8. A system for delivering ultrasound energy to a target region, the system comprising:
an ultrasound transducer comprising a plurality of transducer elements for generating a focal zone of acoustic energy at the target region, wherein at least some of the transducer elements comprise a piezoelectric block having at least one asymmetric feature producing a plurality of natural resonance frequencies;
at least one driver circuit connected to the transducer elements; and
a controller configured to:
(a) determine an optimal sonication frequency for maximizing a peak acoustic intensity in the focal zone; and
(b) based at least in part on the determined optimal sonication frequency, activate the at least one driver circuit to drive the transducer elements with asymmetric features at one or more of the natural resonance frequencies to produce an ultrasound emission having a dominant output frequency corresponding to the optimal sonication frequency.

9. The system of claim 8, further comprising an imaging system for acquiring images of the target region or a non-target region located between the transducer and the target region.

10. The system of claim 9, wherein the imaging system comprises at least one of a computer tomography (CT) device, a magnetic resonance imaging device (MRI), a positron emission tomography (PET) device, a single-photon emission computed tomography (SPECT) device, or an ultrasonography device.

11. The system of claim 9, wherein the controller is further configured to determine, based at least in part on the acquired images, a spatial configuration of the target region with respect to the transducer.

12. The system of claim 11, wherein the spatial configuration comprises at least one of an orientation or a location.

13. The system of claim 11, wherein the controller is further configured to compute a steering angle of the focal zone based at least in part on the spatial configuration of the target region with respect to the transducer.

14. The system of claim 11, wherein the controller is further configured to:
determine a plurality of suboptimal frequencies, each associated with a parameter, wherein (i) a change in the parameter results in a change in the peak acoustic intensity in the focal zone and (ii) the suboptimal frequency corresponds to a maximum of the peak acoustic intensity resulting from changes in the associated parameter; and
determine the optimal sonication frequency based at least in part on the suboptimal frequencies.

15. The system of claim 14, wherein the controller is further configured to assign a weighting factor to each of the suboptimal frequencies and determine the optimal sonication frequency based at least in part on the weighting factors.

16. The system of claim 15, wherein the controller is further configured to assign the weighting factors based on at least one of a first anatomic characteristic of the target region, a second anatomic characteristic of a non-target region located between the transducer and the target region, a steering angle of the focal zone, a contribution of each parameter to the maximum of the peak acoustic intensity, or retrospective data based on study of patients who have undergone ultrasound treatment.

17. The system of claim 16, wherein the first or the second anatomic characteristic comprises at least one of a tissue type, a tissue property, a tissue structure, a tissue thickness or a tissue density.

18. The system of claim 15, wherein the controller is further configured to assign the weighting factors using a machine-learning or evolutionary approach.

19. The system of claim 14, wherein the controller is further configured to determine a second one of the suboptimal frequencies based at least in part on a first one of the suboptimal frequencies.

20. The system of claim 9, wherein the controller is further configured to:
use a physical model to predict a thermal map of the target region and non-target region based at least in part on the acquired images; and
determine the optimal sonication frequency based at least in part on the predicted thermal map.
